(19) United States
(12) Patent Application Publication
(10) Pub. No.: US 2011/0003338 A1
(43) Pub. Date: Jan. 6, 2011

(54) ANTIBODIES WITH ENHANCED ADCC FUNCTION

(76) Inventors: Robert Bayer, San Diego, CA (US); Reed J. Harris, San Mateo, CA (US); Feng Li, Mission Viejo, CA (US); Domingos Ng, San Francisco, CA (US); Efren Pacis, Oceanside, CA (US); Amy Shen, San Mateo, CA (US); Marcella Yu, Medford, MA (US)

Correspondence Address: Arnold & Porter LLP (24126);Attn: SV Docketing Dept., 1400 Page Mill Road, Palo Alto, CA, 94304, US

(21) Appl. No.: 12/922,111

(22) PCT Filed: Mar. 11, 2009
(86) PCT No.: PCT/US09/36855

§ 371 Date: Sep. 10, 2010

(30) Foreign Application Priority Data
Mar. 11, 2008 (US) 61/035692

(51) Int. Cl. C12P 21/06; C12N 5/07

(52) U.S. Cl. 435/69.6; 435/326; 435/328

(57) ABSTRACT

The present invention concerns antibodies enhanced antibody-dependent cell mediated cytotoxicity (ADCC) and method for preparation thereof.

### FIELD OF THE INVENTION

The present invention concerns antibodies enhanced antibody-dependent cell mediated cytotoxicity (ADCC) and method for preparation thereof

### BACKGROUND OF THE INVENTION

Antibody-dependent cell-mediated cytotoxicity (ADCC) is a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. It is known that among antibodies of the human IgG class, the IgG1 subclass has the highest ADCC activity and CDC activity, and currently most of the humanized antibodies in clinical oncological practice, including commercially available HERCEPTIN® (trastuzumab) and RITUXAN® (rituximab), which require high effector functions for the expression of their effects, are antibodies of the human IgG1 subclass.

In order to enhance the potency of therapeutic antibodies, it is often desirable to modify the antibodies with respect to effector function, e.g., so as to enhance antigen-dependent cell-mediated cyotoxicity (ADCC) and/or complement dependent cytotoxicity (CDC) of the antibody. This can be of particular benefit in the oncology field, where therapeutic monoclonal antibodies bind to specific antigens on tumor cells and induce an immune response resulting in destruction of the tumor cell. By enhancing the interaction of IgG with killer cells bearing Fc receptors, these therapeutic antibodies can be made more potent.

Enhancement of effector functions, such as ADCC, may be achieved by various means, including introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

Another approach to enhance the effector function of antbodies, including antibodies of the IgG class, is to engineer the glycosylation pattern of the antibody Fc region. An IgG molecule contains an N-linked oligosaccharide covalently attached at the conserved Asn297 of each of the CH2 domains in the Fc region. The oligosaccharides found in the Fc region of serum IgGs are mostly biantennary glycans of the complex type. A number of antibody glycoforms have been reported as having a positive impact on antibody effector function, including antibody-dependent cell mediated cytotoxicity (ADCC). Thus, glycoengineering of the carbohydrate component of the Fc-part, particularly reducing core fucosylation, has been reported by Shinkawa T, et al., J Biol Chem. 2003; 278:3466-73; Niwa R, et al., Cancer Res 2004; 64:2127-33; Okazaki A, et al., J Mol Biol 2004; 336:1239-49; and Shields R L, et al., J Biol Chem 2002; 277:26733-40.

Antibodies with select glycoforms have been made by a number of means, including the use of glycosylation pathway inhibitors, mutant cell lines that have absent or reduced activity of particular enzymes in the glycosylation pathway, engineered cells with gene expression in the glycosylation pathway either enhanced or knocked out, and in vitro remodeling with glycosidases and glycosyltransferases. Rothman et al., 1989; Molecular Immunology 26: 1113-1123, expressed monoclonal IgG in the presence of the glucosidase inhibitors castanospermine and N-methyldeoxynojirimycin, and the mannosidase I inhibitor deoxymannojirimycin. Umana et al., Nature Biotechnology 1999; 17: 176-180, describe enhanced effector function of a chimeric IgG1 expressed in a CHO cell line expressing GNT-III. Shields et al., 2002; JBC 277:26733-26740, 2002, describe enhanced ADCC in human IgG1 expressed in the Lec13 cell line, which is deficient in its ability to add fucose. Shinkawa et al., 2003; JBC 278: 3466-3473,2003, showed that an anti-CD20 IgG1 expressed in YB2/0 cells showed more than 50-fold higher ADCC using purified human peripheral blood mononuclear cells as effector than those produced by Chinese hamster ovary (CHO) cell lines. Monosaccharide composition and oligosaccharide profiling analysis showed that low fucose (Fuc) content of complex-type oligosaccharides was characteristic in YB2/0-produced IgG1s compared with high Fuc content of CHO-produced IgG1s. Kanda et al., 2006; Glycobiology 17, 104-118, describe enhanced ADCC in rituximab bearing afucosyl complex, afucosyl hybrid, Man5, and Man8,9 glycans. Yamane-Ohnuki et al., Biotechnol Bioeng 2004;87:614-22, achieved a reduction of core fucosylation by recombinant antibody expression in CHO cells lacking core-fucosyl transferase activity, whereas Mori et al., Biotechnol Bioeng 2004;88:901-8, maximized effector functions of expressed antibodies using fucosyl transferase specific short interfering RNA (siRNA).

Antibodies bearing predominantly the Man5 glycoform have been described by Wright and Morrison; 1994, J. Exp. Med. 180:1087-1096; 1998; J. Immunology 160: 3393-3402). The antibodies were expressed in the lec1 cell line, which does not have an active GlcNAc Transferase I. Judging from the biphasic clearance curve in FIG. 8 of the J. Exp. Med. paper, there appears to be at least two distinct populations of antibody with different clearance characteristics. The more rapidly cleared population of IgG is presumably antibody bearing Man7,8,9 glycoforms.

### SUMMARY OF THE INVENTION

In one aspect, the invention concerns a mammalian cell lacking GlcNAc Transferase I activity, engineered to express an antibody or a fragment thereof, or an immunoadhesin or a fragment thereof. In a particular embodiment, the mammalian cell additionally has enhanced α-1,2-mannosidase (also referred to herein as α-mannosidase I) activity.

In another aspect, the invention concerns a mammalian cell, in which GlcNAc Transferase I activity is diminished by RNAi knockdown engineered to express an antibody or a fragment thereof, or an immunoadhesin or a fragment thereof. In a particular embodiment, the mammalian cell additionally has enhanced α-1,2-mannosidase activity.

In another aspect, the invention concerns a mammalian cell, in which GlcNAc Transferase I activity is diminished by RNAi knockdown, sufficient to result in a carbohydrate structure comprising 5% or greater, or 10% or greater, or 20% or greater, or 25% or greater, or 30% or greater, or 35% or greater Man5, Man6 glycans, and which may in addition have enhanced α-1,2 mannosidase activity, engineered to express an antibody or a fragment thereof, or an immunoadhesin or a fragment thereof, wherein said fragment comprises at least one glycosylation site.

In another aspect, the invention concerns a mammalian cell, in which GlcNAc Transferase I activity is diminished by RNAi knockdown of the Golgi UDP-GlcNAc transporter, and which additionally may have enhanced α-1,2 mannosidase activity, engineered to express an antibody or a fragment thereof, or an immunoadhesin or a fragment thereof, wherein the fragment comprises at least one glycosylation site.

In a further aspect, the invention concerns a mammalian cell, in which GlcNAc Transferase I activity is diminished by RNAi knockdown of the Golgi UDP-GlcNAc transporter, and which also has GlcNAc transferase I knocked down by RNAi, engineered to express an antibody or a fragment thereof, or an immunoadhesin or a fragment thereof, wherein the fragment comprises at least one glycosylation site.

In yet another aspect, the invention concerns a method for making an antibody or a fragment thereof, or an immunoadhesin or a fragment thereof, bearing predominantly Man5 glycans, comprising culturing a mammalian cell line according to claim 2 or claim 22 under conditions such that said antibody or a fragment thereof, or an immunoadhesin or a fragment thereof is produced.

In a further aspect, the invention concerns a method for recombinant production of an antibody, an immunoadhesin, or a fragment thereof with a controlled amount of Man5 glycans in the carbohydrate structure thereof, comprising expressing nucleic acid encoding the antibody or antibody fragment in a mammalian cell line which has a diminished GlcNAc Transferase I activity as a result of RNAi knockdown.

In a still further aspect, the invention concerns a method for recombinant production of an antibody, an immunoadhesin, or a fragment thereof, bearing predominantly Man5 glycans in the carbohydrate structure thereof, comprising culturing a mammalian cell line lacking GlcNAc Transferase I activity engineered to express said antibody, immunoadhesin, or fragment thereof in the presence of an α-1,2-mannosidase, or contacting the expressed product with such α-1,2-mannosidase, wherein Man7,8,9 glycans are converted to Man5, 6 glycans.

In a still further aspect, the invention concerns a method for making an antibody or a fragment thereof, or an immunoadhesin or a fragment thereof, bearing 5% or greater, or 10% or greater, or 20% or greater, or 25% or greater, or 30% or greater, or 35% or greater, Man5 glycans, comprising culturing a mammalian cell line according to claim 2 or claim 14 under conditions such that said antibody or a fragment thereof, or an immunoadhesin or a fragment thereof is produced, wherein said fragment comprises at least one glycosylation site.

The invention further concerns a method for recombinant production of an antibody, an immunoadhesin, or a fragment thereof, bearing predominantly Man5 glycans in the carbohydrate structure thereof, comprising culturing a mammalian cell line with diminished GlcNAc Transferase I activity due to RNAi knockdown, engineered to express said antibody, immunoadhesin, or a fragment thereof, in the presence of an α-1,2-mannosidase, or contacting the expressed product with such α-1,2-mannosidase, wherein Man7,8,9 glycans are converted to Man5, 6 glycans.

In another aspect, the invention concerns a method for recombinant production of an antibody, an immunoadhesin, or a fragment thereof, bearing predominantly Man5 glycans in the carbohydrate structure thereof, comprising culturing a mammalian cell line in the presence of a toxic lectin to select for clones with diminished GlcNAc Transferase I activity, engineering one or more of said clones with diminished GlcNAc Transferase I activity to express said antibody, immunoadhesin, or a fragment thereof, in the presence of an α-1,2-mannosidase, or contacting the expressed product with such α-1,2-mannosidase, wherein Man7,8,9 glycans are converted to Man5 glycans, wherein said fragment comprises at least one glycosylation site. In a particular embodiment, the mannosidase is endogenous in the cell used for recombinant production.

In yet another aspect, the invention concerns a method for recombinant production of an antibody, an immunoadhesin, or a fragment thereof, bearing predominantly Man5 glycans in the carbohydrate structure thereof, comprising culturing a mammalian cell line lacking UDP-GlcNAc transporter activity engineered to express said antibody, immunoadhesin, or fragment thereof in the presence of an α-1,2-mannosidase, or contacting the expressed product with such α-1,2-mannosidase, wherein Man7,8,9 glycans are converted to Man5 glycans, wherein said fragment comprises at least one glycosylation site. In a particular embodiment, the mannosidase is endogenous in the cell used for recombinant production.

In all aspect, the mammalian cell line may, for example, be a Chinese Hamster Ovary (CHO) cell line.

In all aspects, the cell lines and methods of the present invention can be used for the production of any antibody, including, without limitation, antibodies of diagnostic or therapeutic interest, such as, antibodies binding to one or more of the following antigens: CD3, CD4, CD8, CD19, CD20, CD22, CD34, CD40, EGF receptor (EGFR, HER1, ErbB1), HER2 (ErbB2), HER3 (ErbB3), HER4 (ErbB4), LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DR5, EGFL7, neuropilins and receptors, netrins and receptors, slit and receptors, sema and receptors, semaphorins and receptors, robo and receptors, and M1.

The antibodies and antibody fragments may be chimeric or humanized, and specifically include chimeric and humanized anti-CD20 antibodies, where, in a specific embodiment, the antibody is rituximab or ocrelizumab.

In another embodiment, the humanized antibody is an anti-HER2, anti-HER1, anti-VEGF or anti-IgE antibody, including, without limitation, trastuzumab, pertuzumab, bevacizumab, ranibizumab, and omalizumab, as well as fragments, variants and derivatives of such antibodies.

Antibody fragments include, for example, complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide, provided that they are glycosylated.

### BRIEF DESCRIPTION OF THE DRAWINGS

FIG. 1 depicts a portion of the N-glycan biosynthetic pathway.

FIG. 2. Plasmid vector used to add N-terminus FLAG® tag to GlcNAc Transferase I (GnT-I) protein (Stratagene).

FIG. 3. Plasmid vector used to express small inhibitory RNA (Ambion, Austin. Tex.). Hairpin sequence disclosed as SEQ ID NO: 10.

FIG. 4. SiRNA probe sequences (SEQ ID NOs: 2-6) and their relative positions (in parentheses) in full length GnT-I gene. Each siRNA probe sequence is underlined (a). The underlined sequence close to BamHI site is complementary to the GnT-I mRNA sequence. The two underlined sequences are complementary to each other resulting in formation of the hairpin loop siRNA.

FIG. 5. Western blot analysis of lysates from the co-transfection of the individual siRNA probes and the FLAG®-tagged GnT-I construct. Five individual siRNA expression constructs in addition to empty vector were transiently co-transfected with FLAG®-tagged GnT-I construct. Cell lysates containing equal amounts of cellular protein were analyzed by Western blot with anti-FLAG® antibody (Sigma MO).

FIG. 6A. Cell line generating ocrelizumab was transiently transfected with siRNA expression plasmids. Cell pellets from each sample condition were collected on day 1, 2 and 5 post transfection, and then mRNA was isolated for TaqMan analysis. GnT-I mRNA expression level of control was set to 100%.

FIG. 6B. Man5 level of day 5 post transfection from each sample transfected with the indicated RNAi vector.

FIG. 7. Transient transfection of scramble and RNAi13 vectors into ocrelizumab-generating cell line for a 14-day experiment. Man5 level of HCCF collected at the indicated culture duration was determined using CE-glycan. Error bar represents standard deviation from duplicate runs.

FIG. 8A. cDNA sequence of CHO α-mannosidase I (SEQ ID NO: 11).

FIG. 8B. Amino acid sequence alignment between CHO (SEQ ID NO: 13) and mouse α-mannosidase I (SEQ ID NOS: 12).

FIG. 8C. Configuration of the SV40GS.CMV.Man1.RNAi13 expression plasmid.

FIG. 9A. Relative GnT-I mRNA level in stable clones determined by TAQMAN® assay. Control represents the GnT-I level in untransfected baseline.

FIG. 9B. Man5 level of stable clones at the end of 14 days production run. The Man5% is determined by CE-glycan analysis.

FIG. 10A. Man5 level at various days of culture duration. The Man5 level was determined by CE-glycan assay, and the errors bars represent standard deviations.

FIG. 10B. Comparison of Man5 level after 22 days culture. Four different osmolality in basal media was tested (300, 330, 360, 400 mOsm). The Man5 level was determined by CE-glycan assay.

FIG. 10C. Man5 level with the addition of MnCl₂ on various days of a total 14 day culture. The Man5 level was determined by CE-glycan assay.

FIG. 10D. Man5 level (CE-glycan assay) of GnT-I knockdown clone 6D at different cell culture conditions. Control represents standard production culture media. High osmo represents increased osmolality to 400 mOsm in basal media. Without Mn represents standard production media which lacks manganese.

FIG. 11. Antibody binding to Fc gamma receptor IIIa-V158. Open circles represent HERCEPTIN® (trastuzumab), open squares represent RITUXAN® (rituximab), open triangles represent anti-receptor antibody with 5% Man5 (7-9% afucosyl glycans), open diamonds represent anti-receptor antibody with 16% Man5 (14.6% afucosyl glycans), and closed circles represent anti-receptor antibody with 62% Man5 (11% afucosyl glycans).

FIG. 12. Antibody binding to Fc gamma receptor IIIa-F158. Open circles represent HERCEPTIN® (trastuzumab), open squares represent RITUXAN® (rituximab) open triangles represent anti-receptor antibody with 5% Man5 (7-9% afucosyl glycans), open diamonds represent anti-receptor antibody with 16% Man5 (14.6% afucosyl glycans), and closed circles represent anti-receptor antibody with 62% Man5 (11% afucosyl glycans).

### DETAILED DESCRIPTION OF THE INVENTION

I. Definitions

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and Fcγ□RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)) and mediates slower catabolism, thus longer half-life.

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods 202:163 (1996), may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

The term “monoclonal antibody” is used to refer to an antibody molecule synthesized by a single clone. The modifier “monoclonal” indicates the character of the antibody as being obtained from a substantially homogeneous population of antibodies, and is not to be construed as requiring production of the antibody by any particular method. Thus, monoclonal antibodies may be made by the hybridoma method first described by Kohler and Milstein, Nature 256:495 (1975); Eur. J. Immunol. 6:511 (1976), by recombinant DNA techniques, or may also be isolated from phage or other antibody libraries.

The term “polyclonal antibody” is used to refer to a population of antibody molecules synthesized by a population of B cells.

“Antibody fragments” comprise a portion of a full length antibody, generally the antigen binding domain(s) or variable domain(s) thereof. Examples of antibody fragments include, but are not limited to, Fab, Fab′, F(ab′)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide. Specifically within the scope of the invention are bispecific antibody fragments.

Antibodies are glycoproteins, with glycosylation in the Fc region. Thus, for example, the Fc region of an IgG immunoglobulin is a homodimer comprising interchain disulfide-bonded hinge regions, glycosylated CH2 domains bearing N-linked oligosaccharides at asparagine 297 (Asn-297), and non-covalently paired CH3 domains. Glycosylation plays an important role in effector mechanisms mediated FcγRI, FcγRII, FcγRIII, and C1q. Thus, antibody fragments of the present invention must include a glycosylated Fc region and an antigen-binding region.

The terms “bispecific antibody” and “bispecific antibody fragment” are used herein to refer to antibodies or antibody fragments with binding specificity for at least two targets. If desired, multi-specificity can be combined by multi-valency in order to produce multivalent bispecific antibodies that possess more than one binding site for each of their targets. For example, by dimerizing two scFv fusions via the helix-turn-helix motif, (scFv)₁-hinge-helix-turn-helix-(scFv)₂, a tetravalent bispecific miniantibody was produced (Müller et al., FEBS Lett. 432 (1-2):45-9 (1998)). The so-called ‘di-bi-miniantibody’ possesses two binding sites to each of it target antigens.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, Vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “naked antibody” is an antibody (as herein defined) that is not conjugated to a heterologous molecule, such as a cytotoxic moiety or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified to greater than 95% by weight of antibody as determined by non-reducing SDS-PAGE, CE-SDS, or Bioanalyzer. Isolated antibody includes the antibody in situ within recombinant cells since at least one component of the antibody's natural environment will not be present. Ordinarily, however, isolated antibody will be prepared by at least one purification step.

As used herein, the term “immunoadhesin” designates antibody-like molecules which combine the “binding domain” of a heterologous protein (an “adhesin”, e.g. a receptor, ligand or enzyme) with the effector functions of immunoglobulin constant domains. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The immunoglobulin constant domain sequence in the immunoadhesin may be obtained from any immunoglobulin, such as IgG1, IgG.2, IgG3, or IgG4 subtypes, IgA, IgE, IgD or IgM. For further details of immunoadhesins, ligand binding domains and receptor binding domains see, e.g. U.S. Pat. Nos. 5,116,964; 5,714,147; and 6,406,604, the disclosures of which are hereby expressly incorporated by reference.

II. Detailed Description

The present invention provides a method for preparing antibodies and antibody-like molecules, such as Fc fusion proteins (immunoadhesins), bearing predominantly Man5 glycans, but with decreased amounts of Man7, Mang, and Man9, in a mammalian host cell, by manipulating the glycosylation machinery of the recombinant mammalian host cell producing the antibody or antibody-like molecule.

General Methods for the Recombinant Production of Antibodies

The antibodies and other recombinant proteins herein can be produced by well known techniques of recombinant DNA technology. Thus, aside from the antibodies specifically identified herein, the skilled practitioner could generate antibodies directed against an antigen of interest, e.g., using the techniques described below.

The antibodies produced in accordance with the present invention are directed against an antigen of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. receptor) or ligand such as a growth factor. Exemplary molecular targets for antibodies encompassed by the present invention include CD proteins such as CD3, CD4, CD8, CD19, CD20, CD22, CD34, CD40; members of the ErbB receptor family such as the EGF receptor (EGFR, HER1, ErbB1), HER2 (ErbB2), HER3 (ErbB3) or HER4 (ErbB4) receptor; cell adhesion molecules such as LFA-1, Mac1, p150,95, VLA-4, ICAM-1, VCAM and αv/β3 integrin including either α or β subunits thereof (e.g. anti-CD11a, anti-CD18 or anti-CD11b antibodies); growth factors such as VEGF; IgE; blood group antigens; flk2/flt3 receptor; obesity (OB) receptor; mpl receptor; CTLA-4; protein C, neutropilins and receptors, EGF-C, ephrins and receptors, netrins and receptors, slit and receptors, anti-M1, or any of the other antigens mentioned herein. Antigens to which the antibodies listed above bind are specifically included within the scope herein.

For recombinant production of the antibody, the nucleic acid encoding it may be isolated and inserted into a replicable vector for further cloning (amplification of the DNA) or for expression. In another embodiment, the antibody may be produced by homologous recombination, e.g. as described in U.S. Pat. No. 5,204,244, specifically incorporated herein by reference. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence, e.g., as described in U.S. Pat. No. 5,534,615 issued Jul. 9, 1996 and specifically incorporated herein by reference.

The antibodies of the present invention must be glycosylated, and thus suitable host cells for cloning or expressing the DNA encoding antibody chains or other antibody-like molecules include mammalian host cells. Interest has been great in mammalian host cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with expression or cloning vectors for antibody production and cultured in conventional nutrient media modified as appropriate for inducing promoters, selecting transformants, or amplifying the genes encoding the desired sequences.

The mammalian host cells may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The antibody composition prepared from the cells can be purified using, for example, hydroxylapatite chromatography, ion exchange chromatography, gel electrophoresis, dialysis, and affinity chromatography, with affinity chromatography being the primary purification step. The suitability of protein A as an affinity ligand depends on the species and isotype of any immunoglobulin Fc domain that is present in the antibody. Protein A can be used to purify antibodies that are based on human γ1, human γ2, or human γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3domain, the BAKERBOND ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin, chromatofocusing, SDS-PAGE, hydrophobic interaction chromatography, and ammonium sulfate precipitation are also available depending on the antibody to be recovered.

Following any preliminary purification step(s), the mixture comprising the antibody of interest and contaminants may be subjected to additional purification steps to achieve the desired level of purity.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human FR for the humanized antibody (Sims et al., J. Immunol., 151:2296 (1993)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immnol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to another approach described in WO96/27011, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Antibodies with more than two valencies are contemplated. For example, trispecific antibodies can be prepared. Tutt et al. J. Immunol. 147: 60 (1991).

Immunoadhesins

The simplest and most straightforward immunoadhesin design combines the binding domain(s) of the adhesin (e.g. the extracellular domain (ECD) of a receptor) with the hinge and Fc regions of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc domain of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Just as the antibodies and antibody fragments, the immunoadhesin structures of the present invention must have an Fc region. Various exemplary assembled immunoadhesins within the scope herein are schematically diagrammed below:

AC_{H}-(AC_{H}, AC_{L}-AC_{H}, AC_{L}-V_{H}C_{H}, or V_{L}C_{L}-AC_{H});

AC_{L}-AC_{H}-(AC_{L}-AC_{H}, AC_{L}-V_{H}C_{H}, V_{L}C_{L}-AC_{H}, or V_{L}C_{L}-V_{H}C_{H})

AC_{L}-V_{H}C_{H}-(AC_{H}, or AC_{L}-V_{H}C_{H}, or V_{L}C_{L}-AC_{H});

V_{L}C_{L}-AC_{H}-(AC_{L}-V_{H}C_{H}, or V_{L}C_{L}-AC_{H}); and

(A-Y)ₙ-(V_{L}C_{L}-V_{H}C_{H})₂,
wherein each A represents identical or different adhesin amino acid sequences;
V_{L} is an immunoglobulin light chain variable domain;
V_{H} is an immunoglobulin heavy chain variable domain;
C_{L} is an immunoglobulin light chain constant domain;
C_{H} is an immunoglobulin heavy chain constant domain;
n is an integer greater than 1;
Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom, et al., Mol. Immunol. 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell 61:1303-1313 (1990); and Stamenkovic et al., Cell 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

Antibodies with Enhanced ADCC Function

Following the expression of proteins in eukaryotic, e.g. mammalian host cells, the proteins undergo post-translational modifications, often including the enzymatic addition of sugar residues, generally referred to as “glycosylation”.

Glycosylation of polypeptides is typically either N-linked or O-linked. N-linked refers to the attachment of the carbohydrate moiety to the side-chain of an asparagine residue. The tripeptide sequences, asparagine (Asn)-X-serine (Ser) and asparagine (Asn)-X-threonine (Thr), wherein X is any amino acid except proline, are recognition sequences for enzymatic attachment of the carbohydrate moiety to the asparagine side chain. O-linked glycosylation refers to the attachment of one of the sugars N-acetylgalactosamine, galactose, fucose, N-acetylglucosamine, or xylose to a hydroxyamino acid, most commonly serine or threonine, although 5-hydroxyproline or 5-hydroxylysine may also be involved in O-linked glycosylation.

Glycosylation patterns for proteins produced by mammals are described in detail in The Plasma Proteins: Structure, Function and Genetic Control, Putnam, F. W., ed., 2nd edition, Vol. 4, Academic Press, New York, 1984, especially pp. 271-315. In this chapter, asparagine-linked oligosaccharides are discussed, including their subdivision into a least three groups referred to as complex, high mannose, and hybrid structures, as well as glycosidically linked oligosaccharides.

In the case of N-linked glycans, there is an amide bond connecting the anomeric carbon (C-1) of a reducing-terminal N-acetylglucosamine (GlcNAc) residue of the oligosaccharide and a nitrogen of an asparagine (Asn) residue of the polypeptide. In animal cells, O-linked glycans are attached via a glycosidic bond between N-acetylgalactosamine (GalNAc), galactose (Gal), fucose, N-acetylglucosamine, or xylose and one of several hydroxyamino acids, most commonly serine (Ser) or threonine (Thr), but also hydroxyproline or hydroxylsine in some cases.

The biosynthetic pathway of O-linked oligosaccharides consists of a step-by-step transfer of single sugar residues from nucleotide sugars by a series of specific glycosyltransferases. The nucleotide sugars which function as the monosaccharide donors are uridine-diphospho-GalNAc (UDP-GalNAc), UDP-GlcNAc, UDP-Gal, guanidine-diphospho-fucose (GDP-Fuc), and cytidine-monophospho-sialic acid (CMP-SA).

In N-linked oligosaccharide synthesis, initiation of N-linked oligosaccharide assembly does not occur directly on the Asn residues of the protein, but involves preassembly of a lipid-linked precursor oligosaccharide which is then transferred to the protein during or very soon after its translation from mRNA. This precursor oligosaccharide (Glc₃Man₉GlcNAc₂) is synthesized while attached via a pyrophosphate bridge to a polyisoprenoid carrier lipid, a dolichol, with the aid of a number of membrane-bound glycosyltransferases. After assembly of the lipid-linked precursor is complete, another membrane-bound enzyme transfers it to sterically accessible Asn residues which occur as part of the sequence -Asn-X-Ser/Thr-.

Glycosylated Asn residues of newly-synthesized glycoproteins transiently carry only one type of oligosaccharide, Glc₃Man₉GlcNAc₂. Processing of this oligosaccharide structure generates the great diversity of structures found on mature glycoproteins.

The processing of N-linked oligosaccharides is accomplished by the sequential action of a number of membrane-bound enzymes and includes removal of the three glucose residues, removal of a variable number of mannose residues, and addition of various sugar residues to the resulting trimmed core.

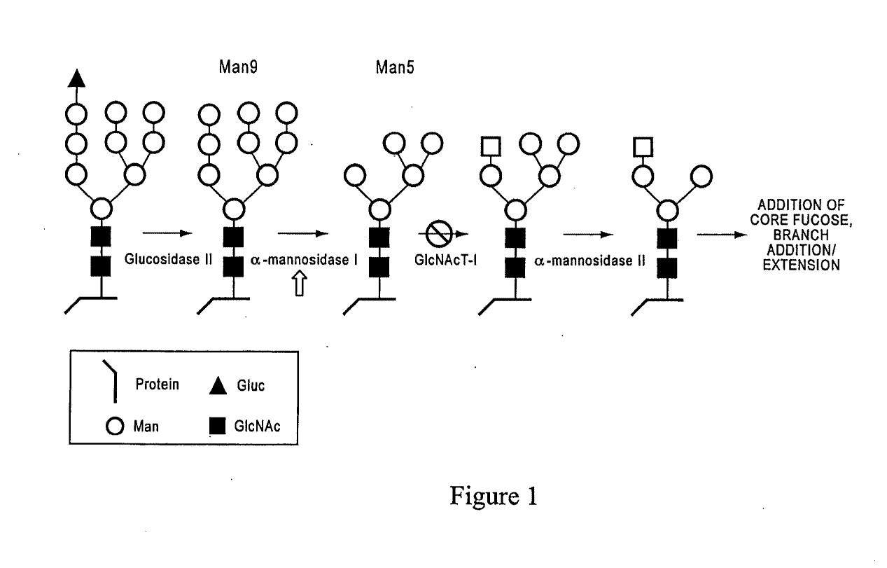

A part of the N-glycan biosynthetic pathway is shown in FIG. 1.

Four of the mannose residues of the Man₉GlcNAc₂ moiety can be removed by α-mannosidase Ito generate N-linked Man₅₋₉GlcNAc₂, all of which are commonly found on vertebrate glycoproteins. As shown in FIG. 1, the Man₅GlcNAc₂ can serve as a substrate for GlcNAc transferase I (GlcNAcT-I), which transfers a β1→2-linked GlcNAc residue from UDP-GlcNAc to the α1→3-linked mannose residue to form GlcNAcMan₅GlcNAc₂, which is further trimmed by α-mannosidase II, which removes two mannose residues to generate a protein-linked oligosaccharide with the composition GlcNAcMan₃GlcNAc₂. This structure is a substrate for GlcNAc transferase II (not shown).

This stage is followed by a complex series of processing steps, including sequential addition of monosaccharides to the oligosaccharide chain by a series of membrane-bound glycosyltransferases, which differ between various cell types. As a result, a diverse family of “complex” oligosaccharides is produced, including various branched, such as biantennary (two branches), triantennary (three branches) or tetraantennary (four branches) structures.

A number of antibody glycoforms have been reported as having a positive impact on antibody effector function, including antibody-dependent cell mediated cytotoxicity (ADCC). This can be of particular benefit in the oncology field, where therapeutic monoclonal antibodies bind to specific antigens on tumor cells and induce an immune response resulting in destruction of the tumor cell. By enhancing the interaction of IgG with killer cells bearing Fc receptors, these therapeutic antibodies can be made more potent.

The present invention discloses methods for producing antibodies having an increased amount of the Man5 glycoform while diminishing the amount of Man7,8,9 relative to what has been previously described. It also describes a method for modulating the amount of the Man5 glycoform produced.

As discussed above, in the N-glycan biosynthetic pathway, a portion of which is depicted in FIG. 1, GlcNAc Transferase I adds a GlcNac moiety to the terminal α-1,3 arm of Man5, which can then be acted on by α-mannosidase II. By abrogating or modulating the activity of GlcNAc Transferase I, the proportion of antibodies bearing Man5 glycans can be increased.

The amount of Man7,8,9 glycoforms can be diminished by enhancing α-1,2 mannosidase activity. By the use of an α-1,2 mannosidase either in vivo or in vitro, the more rapidly cleared Man7,8,9 glycans can be converted to Man5.

The present invention also provides a method for producing antibodies with a variable amount of Man5 using RNA interference (RNAi) knockdown.

RNA interference (RNAi) is a method for regulating gene expression. RNA molecules can bind to single-stranded mRNA molecules with a complementary sequence and repress translation of particular genes. The RNA can be introduced exogenously (small interfering RNA, or siRNA), or endogenously by RNA producing genes (micro RNA, or miRNA). For example, double-stranded RNA complementary to GlcNAc Transferase I can decrease the amount of this glycosyltransferase expressed in an antibody expressing cell line, resulting in an increased level of the Man5 glycoform in the antibody produced. Unlike in gene knockouts, where the level of expression of the targeted gene is reduced to zero, by using different fragments of the particular gene, the amount of inhibition can vary, and a particular fragment may be employed to produce an optimal amount of the desired glycoform. An optimal level can be determined by methods well known in the art, including in vivo and in vitro assays for Fc receptor binding, effector function including ADCC, efficacy, and toxicity. The use of the RNAi knockdown approach, rather than a complete knockout, allows the fine tuning of th amount of Man5 glycan to an optimal level, which may be of great benefit, if the production of antibodies bearing less than 100% Man5 glycans is desirable.

The α-1,2 mannosidase activity can be enhanced in a variety of ways. For example, α-1,2 mannosidase activity can be enhanced by providing additional copies of the α-mannosidase I present in the recombinant host cell used for antibody production.

In other embodiments, an α-1,2 mannosidase from a microbial cell line may be transfected into the expressing cell line. Alpha-1,2-mannosidase from different species have different specificity toward the various high mannose glycans. A commercially available α-mannosidase I, α-1,2-mannosidase from Aspergillus saitoi, has demonstrated robust in vitro trimming of highly-enriched Man9 glycoform to Man5. Contreras et. al. have showed that the α-1,2-mannosidase from Trichoderma reesei alone can trim all four mannoses from Man9 to yield homogenous Man5 glycan (Maras et al., J. Biotechnol., 77: 255-263 (2000); Petegem et al., J. Mol. Biol., 312: 157-165 (2001)). The A. Saitoi or T. reesei α-1,2-mannosidases can be used with the protein A-purified ocrelizumab with high level of Man 9 as a substrate.

In another embodiment, an α-1,2 mannosidase from other mammalian species may be transfected into the expressing cell line.

It is also apparent in higher organisms that different endogenous mannosidases are involved in the trimming of each mannose to convert Man9 to Man5. In fact, most species utilize two mannosidases, one in the endoplasmic reticulum(ER) and another one in the golgi apparatus, to trim Man9 to Man5 in a two-step reaction (Gonzalez et al., J. Biol. Chem., 274 (30): 21375-21386 (1999); Mast and Moremen, Methods Enzymol., 415: 31-46 (2006)). The two step processing is discussed in the paper by Ichishima et al. (Ichishima et al., Biochem. J., 339: 589-597 (1999)). Man8B appears to be the optimal intermediate which has the highest probability to be converted to Man5 using a Golgi mannosidase. Many ER mannosidases have been identified to successfully convert Man9 to Man8B (Gonzalez et al., J. Biol. Chem., 274 (30): 21375-21386 (1999); Jelinek-Kelly and Herscovics, J. Biol. Chem., 263 (29): 14757-14763 (1988)), which, in alternative embodiments, can subsequently be trimmed to Man5 using either the α-1,2-mannosidase from Aspergillus saitoi or Trichoderma reesei.

Another approach toward generating homogenous Man5 glycoform involves combining the RNA interference technology and the in vitro trimming reaction discussed above. Since CHO cells use two mannosidases to convert Man9 to Man5, the CHO golgi mannosidase can be knocked-down using RNAi which would lead to the accumulation of Man8B. The Man8B-enriched antibodies can subsequently be purified, and then converted to Man5 by the same in vitro trimming reaction using α-1,2-mannosidase from Aspergillus saitoi or Trichoderma reesei. Alternatively, the in vitro trimming reaction may be incorporated in vivo by expressing the α-1,2-mannosidase in the same cell line where the CHO golgi mannosidase is knockdown specifically. This will eliminate a purification step prior to the conversion from Man8B to Man5.

In yet another embodiment, any of the previously described mannosidases may be used post expression in vitro to trim Man6,7,8,9 to Man5.

The following examples are offered for illustrative purposes only, and are not intended to limit the scope of the present invention in any way.

All patent and literature references cited in the present specification are hereby incorporated by reference in their entirety.

### EXAMPLE 1

### Knock Down of N-acetylglucosaminyl Transferase I (GnT-I) by Small Inhibitory RNA (siRNA)

Cloning of GnT-I cDNA and FLAG® Tagging of Isolated cDNA:

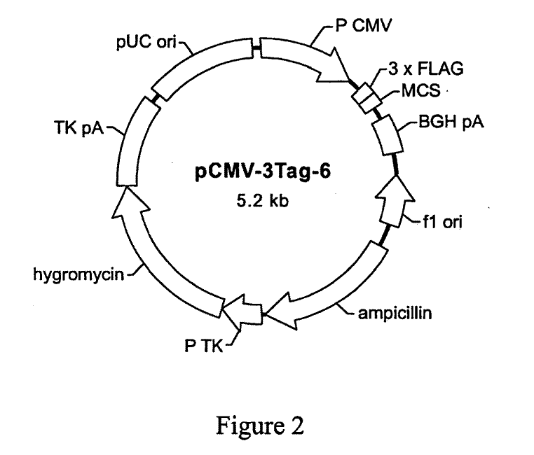

In order to obtain antibodies with oligomannose-type glycans in CHO cells, an RNAi approach was employed to knock down the expression of the endogenous GnT-I gene. A 1.3 kb fragment of GnT-I coding sequence (NCBI Accession No: U65791) was cloned by reverse transcription polymerase chain reaction (RT-PCR) using total RNA purified from CHO DP12 cells. The PCR fragment was then cloned into pCMV-3Tag-6 vector (Cat # 240195) from Strategene (FIG. 2). The DNA sequence encoding the full-length GnT-I protein was cloned in the BamHI and HindIII sites. Three copies of FLAG® tag (MetAspTyrLysAspAspAspAspLys) (SEQ ID NO: 1) were fused to the 5′ end of the isolated GnT-I cDNA sequence for western blot analysis with anti-FLAG® antibody.

Small Inhibitory RNA (siRNA) Probe Design and Cloning into the Expression Vector:

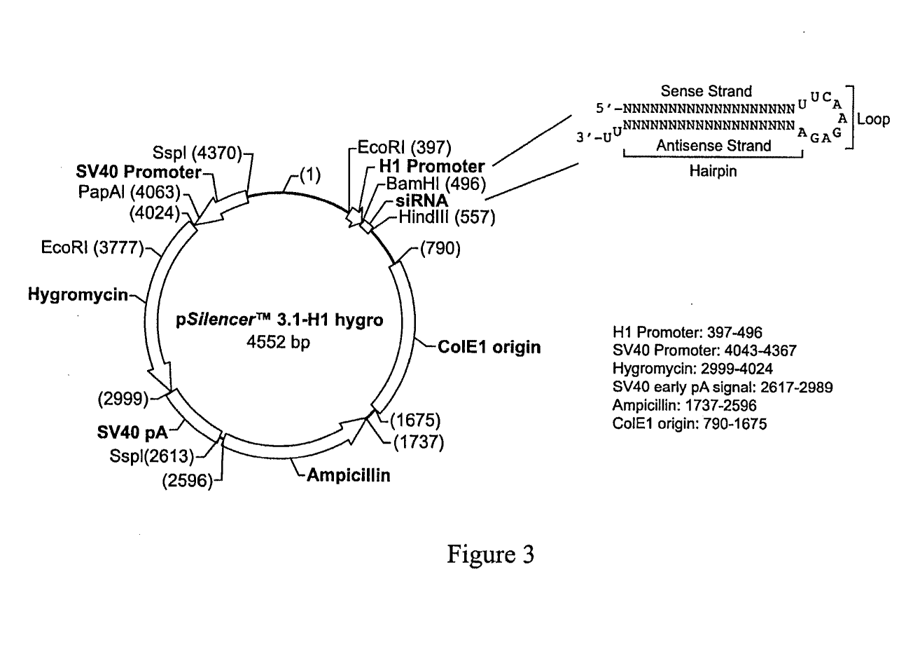

The method used to design 5 siRNA probes (SEQ ID NOs: 2-6) to target the CHO GnT-I gene was described by Elbashir et al, Methods 26 (2):199-213 (2002). The siRNA probes were constructed using annealed synthetic oligonucleotides independently cloned into the pSilencer 3.1-H1 hygro plasmid (FIG. 3) from Ambion, Inc. (Austin, Tex.) to produce short hairpin siRNAs. The DNA sequences encoding siRNA probes were cloned into BamHI and HindIII sites under the control of PolIII type H1 promoter. The transcript from H1 promoter forms a hairpin-loop siRNA, consisting of a 19 nucleotide sense sequence specific to the GnT-I gene, linked to its reverse complement antisense sequence by a 9 nucleotide hairpin-look sequence.

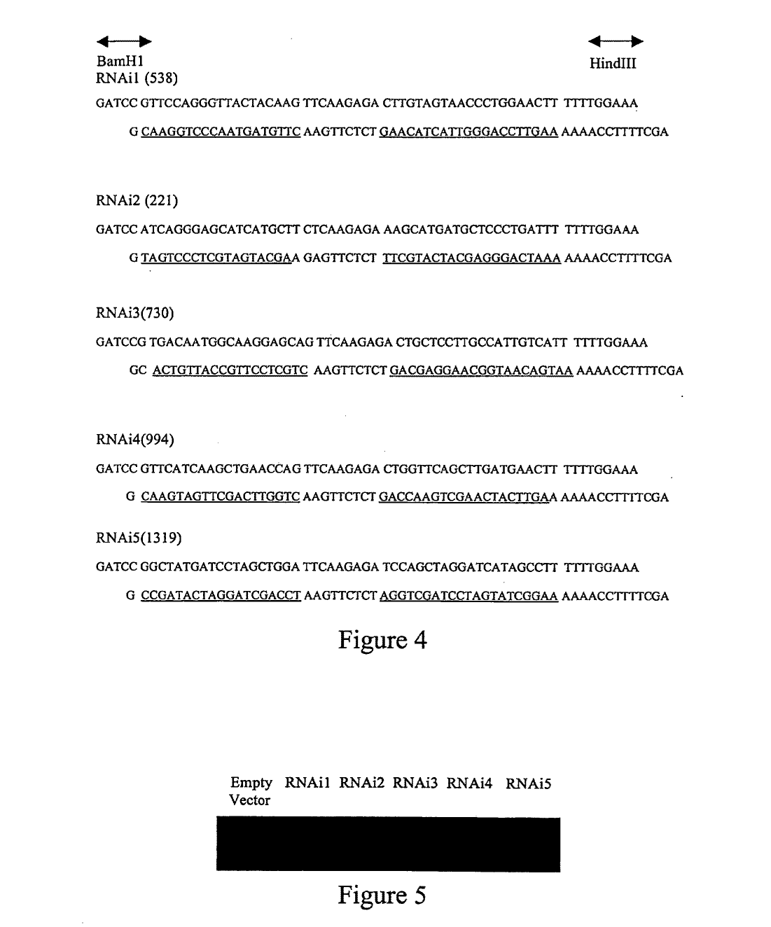

Each siRNA probe consisted of a 19 nucleotide sense sequence specific to the GnT-I gene, linked to its reverse complement anti-sense sequence by a 9 nucleotide hairpin-loop sequence and followed by 5 6U's at the 3′ end (FIG. 3). FIG. 4 shows the 5 siRNA sequences targeting the GnT-I gene. The ability of these siRNA probes to cleave the GnT-I transcript was tested by transient cotransfection of each siRNA expression probe plasmid with the FLAG®-tagged GnT-I plasmids into CHO cells. An empty pSilencer (Ambion, Inc.) vector plasmid, which served as a negative control, was also cotransfected with the FLAG®-tagged GnT-I plasmids. Cells were lysed extracted 24 hours after transfection and the cell lysate was analyzed by western blot with anti-FLAG®M2 antibody (Sigma, Mo.). As expected, the control plasmid did not inhibit expression of FLAG-tagged GnT-I, whereas the siRNA probes had various degrees of inhibition on FLAG®-tagged GnT-I fusion protein expression (FIG. 5). RNAi1 and RNAi3, which demonstrated markedly stronger inhibitory effects than the rest of the RNAi's, were chosen for further evaluation.

Transient Expression of siRNA Expression Plasmids into Cell Line Generating Ocrelizumab

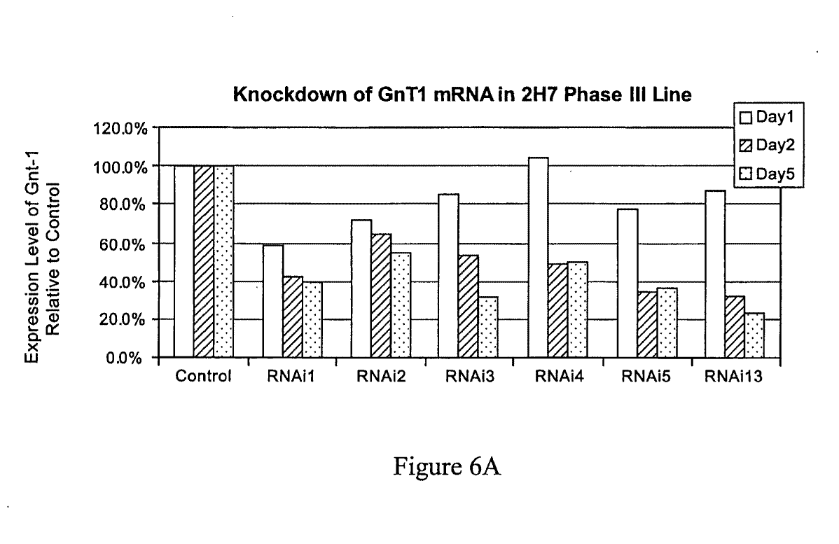

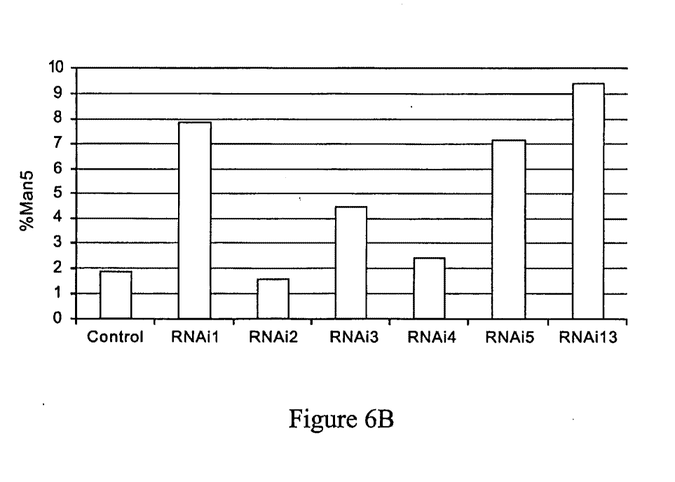

The 5 siRNA expression plasmids (RNAi1, RNAi2, RNAi3, RNAi4, and RNAi5) along with a combination siRNA plasmid containing the sequences of RNAi1 and RNAi3 (RNAi13) were transiently transfected into the cell line for ocrelizumab production. As a control, a scrambled plasmid which contains a random mouse sequence with no homology to GnT-I or any known genes was transfected in parallel. The transfection method followed a standard serum containing transient transfection protocol with LIPOFECTAMINE™ 2000. Briefly, on the day of transfection, cells were seeded at 1.5×10⁶ cells/mL in non-selective growth media in the presence of fetal bovine serum (FBS). DNA and LIPOFECTAMINE™ were added to transfection media in separate tubes and subsequently mixed and incubated at room temperature for 30 minutes. The DNA complex was then added to the cell culture. 24 hours later, transfected culture was media exchanged into production media. Harvested cell culture fluid (HCCF) and cell pellets were collected on days 1, 2 and 5 post transfection. HCCF was analyzed using a CE-glycan assay to determine levels of different glycoforms, and cell pellets were used for quantitative qPCR analysis to measure the endogenous mRNA level of GnT-I. To perform qPCR, mRNA was isolated by RNeasy® 96 well kit (Qiagen) or MagMAX™-96 total RNA isolation kit (Ambion). A TAQMAN® analysis was performed to measure GnT-I mRNA expression level during the course of the experiment (FIG. 6A). The sequences of the primers and probe, which cover the 3′ end of the cDNA (bp1260-1324) are as follows:

| Forward primer | |
|---|---|
| CGTTGTCACTTTCCAGTTCAG | (SEQ ID NO: 7) |
| Reverse Primer | |
| AGCCTTCCCAGGTTTGTG | (SEQ ID NO: 8) |
| Probe | |
| FAM-ACGTGTCCACCTGGCACCCC-TAMRA | (SEQ ID NO: 9) |

The mRNA analysis shown in FIG. 6A demonstrates that all RNAi plasmids targeting GnT-I were able to knock down GnT-I mRNA significantly, with a maximum of 80% knockdown compared to control (transfected with scramble plasmid) 5 days post transfection. GnT-I expression level of control was set to 100%. Knockdown activity in the TAQMAN® assay correlated well with western blot analysis of FLAG®-tagged GnT-I, in which RNAi1 and RNAi3 seemed to be the strongest inhibitors in both assays. RNAi13 provided additional inhibition compared to RNAi1 and RNAi3 individually, and was chosen to be the primary RNAi vector for all subsequent studies.

### EXAMPLE 2

### Measuring Man5 Level of Antibodies

To determine the actual Man5 level of the antibodies collected in HCCF, capillary electrophoresis, referred to as “CE-glycan”, was selected to be the standard method to measure released glycans from the antibody. Briefly, the antibodies from HCCF were purified using a preparative protein-A purification method. Then the N-linked glycan attached to the Fc region is cleaved off by peptide-N-glycosidase F (PNGase F) with an overnight incubation at 37° C. The protein was precipitated after the reaction to separate it from the cleaved glycans, which were then labeled with 8-aminopyrene-1,3,6-trisulfonate (APTS) by reductive amination. The labeled glycans were then analyzed using capillary electrophoresis against APTS-labeled glycan standards with specific elution profile. The details of the assay can be found on the Beckman Coulter website. The Man5 content of the antibodies assayed at Day 5 correlated well with TAQMAN® data (FIG. 6A), with RNAi13 having the highest Man5 content at approximately 9% (FIG. 6B).

Man5 Level Stable During 14 Day Run of Transient Transfection Experiment.

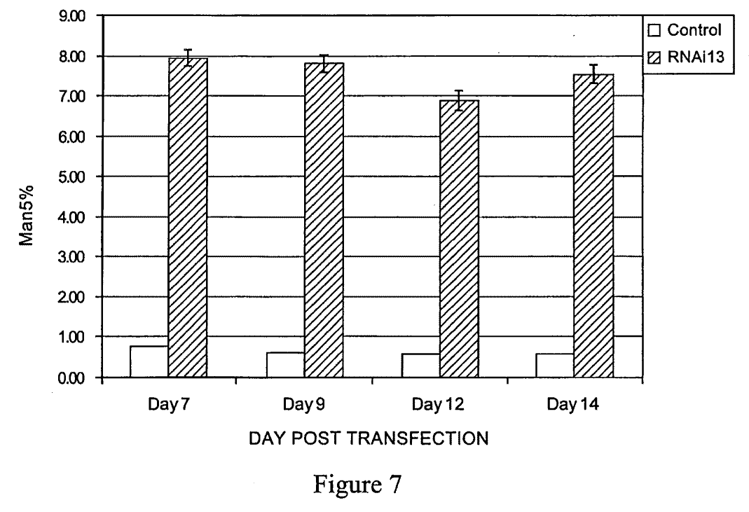

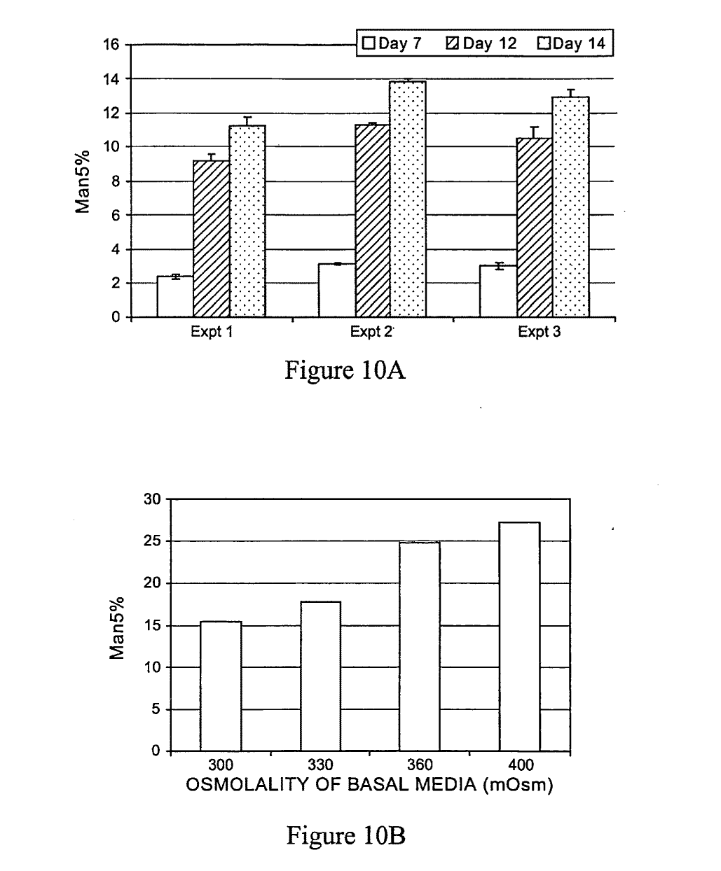

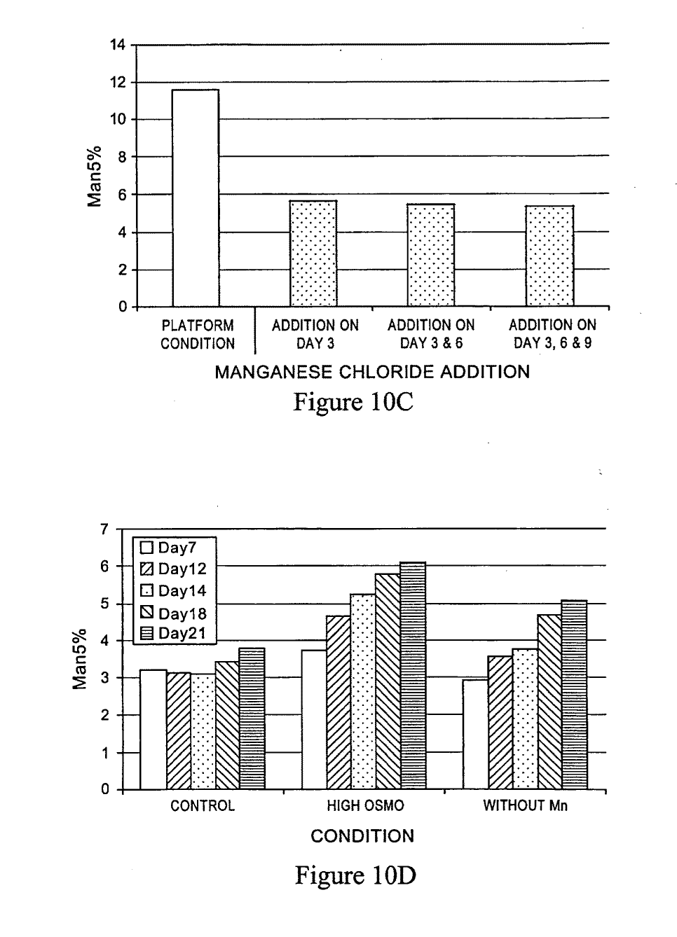

In order to increase the Man5 level with transient expression of the RNAi13 plasmid, longer cell culture duration was tested in the same cell line (up to 14 days). Experience with other antibodies indicated that there was an increased Man5 level with increased production culture duration (FIG. 10A). A similar transient transfection protocol was used in the 14-day experiment. The cell line was transfected with scrambled or RNAi13 vectors using LIPOFECTAMINE™. HCCF was collected at various day post transfection, and samples were analyzed using a CE-glycan assay to determine the Man5 level. FIG. 7 shows the Man5 level at the indicated culture duration, with the RNAi13 plasmid resulted in roughly 10-fold higher Man5 level than the control condition, and the level appeared to be stable throughout the entire run. In addition, the GnT-I mRNA level for this particular experiment was similar to the 5 day culture (data not shown).

### EXAMPLE 3

### Cloning of CHO α-mannosidase I cDNA

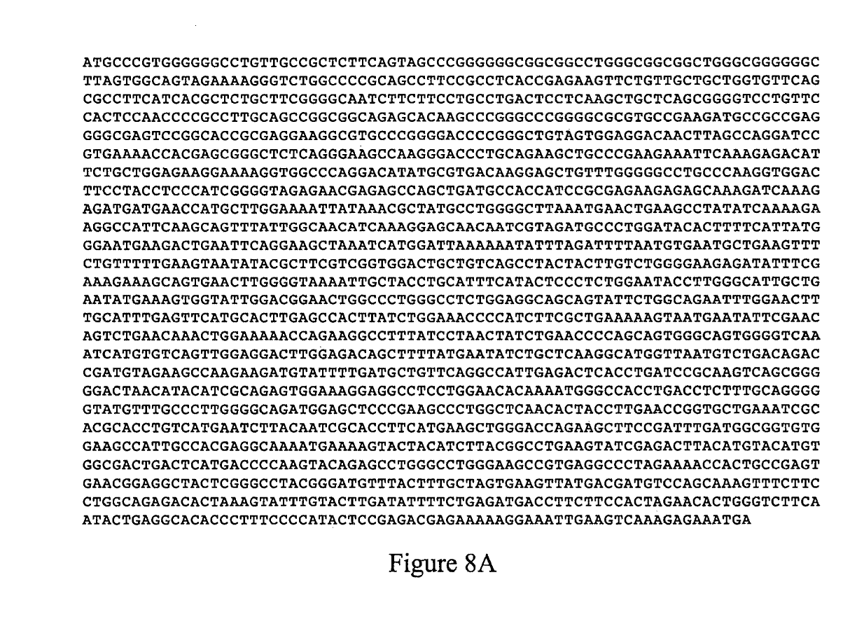

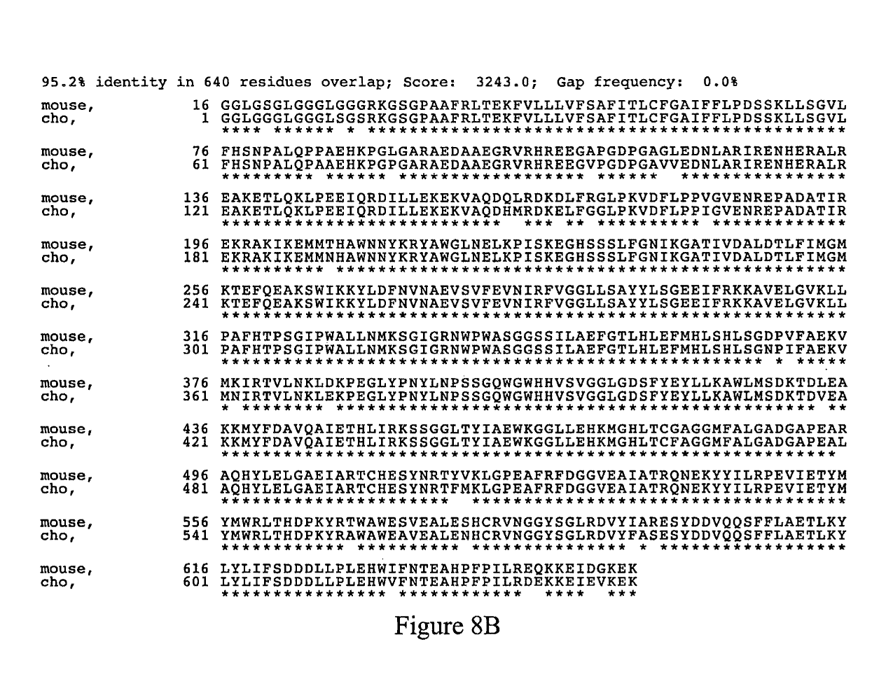

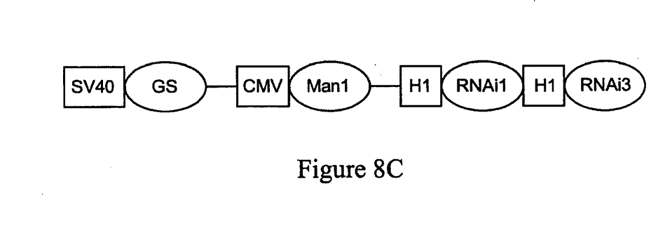

The same total RNA used to clone GnT-I as described above was also used to clone CHO α-mannosidase I. α-mannosidase I is another important enzyme in the glycosylation pathway. It is responsible for converting the high mannose structures Man7,8,9 into Man5,6. By overexpressing this protein, it could potentially result in a more uniform conversion to Man5. First, coding sequences of homologue from homo sapien, Mus musculus, Rattus norvegecus were aligned to uncover conserved regions that could be used to clone out the CHO gene. A conserved area upstream of the 5′ end of the coding sequence and a small region after the stop codon was cloned out the CHO α-mannosidase I. The cDNA has a size of 1.9 kB (FIG. 8A). When alignment was done on the protein level, there was significantly high homology (95%) between the mannosidases from mouse and CHO cells based on amino acid sequences (FIG. 8B). The cDNA of the CHO α-mannosidase I and the GnT-I RNAi13 cassette were cloned into another expression vector SV40.GS.CMV.nbe (FIG. 8C).

### EXAMPLE 4

### Stable Cell Line Development to Express shRNA for Constant Knockdown of GnT-I

Transient transfection of RNAi13 vector into ocrelizumab resulted in a roughly 10-fold increase in Man5 levels, from 0.5-1% to 9%. In effort to further increase the Man5 level, stable cell line development was undertaken to create stable clones with the shRNA incorporated into the genome and therefore is expected to provide a stable expression level to knockdown GnT-I in a more consistent fashion. The standard protocol for developing stable cell clones was done with the RNAi13 plasmid (Shen et al. (2007), Metabolic engineering to control glycosylation In M. Butler (Ed.), Cell culture and Upstream Processing (pp. 131-148). New York, N.Y.: Taylor & Francis Group), and hygromycin selection was used due to the resistance gene present on the vector (FIG. 3). In short, transfection was done in the same fashion as transient transfection experiment using LIPOFECTAMINE™. Instead of being exchanged into production media 24 hours post transfection, the cells were exchanged into selection media containing 0.5 mg/mL hygromycin selective pressure, and then plated onto petri dishes at various seeding densities. The dishes (20-50 dishes total) were incubated in a CO₂ humidified incubator at 37 ° C. for 2-3 weeks until clones were observed. The individual clones were transferred into 96-well plates (1 clone/well), and approximately 200-300 clones were picked at the first stage. In order to select clones with a potentially high Man5 level, GnT-I mRNA level of all clones were determined using TAQMAN® assay to select for clones with lowest GnT-I mRNA level. Subsequently, selected clones were scaled up to 48-well plate, 24-well plate, 6-well plate, T75 cultures flask, and then finally shake flasks. Roughly 12 clones were selected to perform an initial production culture, which is a 14 day culture in production media with the addition of 10% nutrient supplement on day 3. The top clones with the highest amount of Man5 were banked and stored for future use.

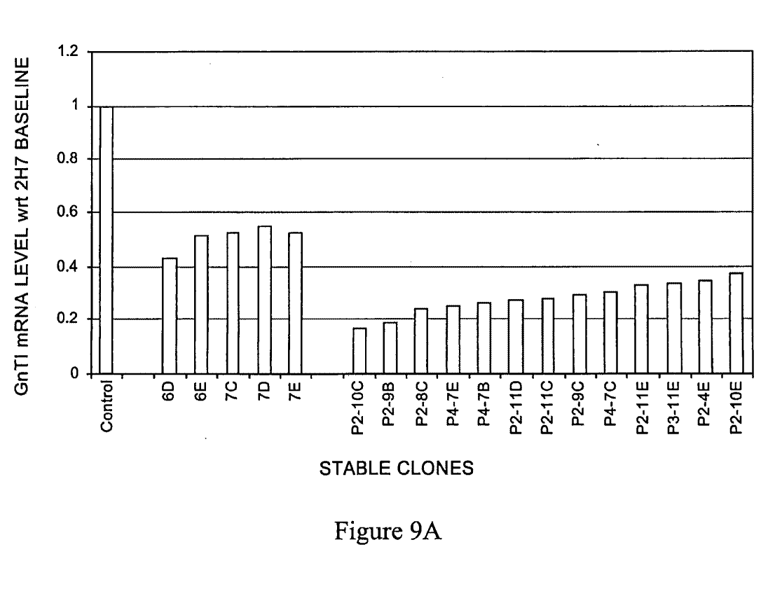

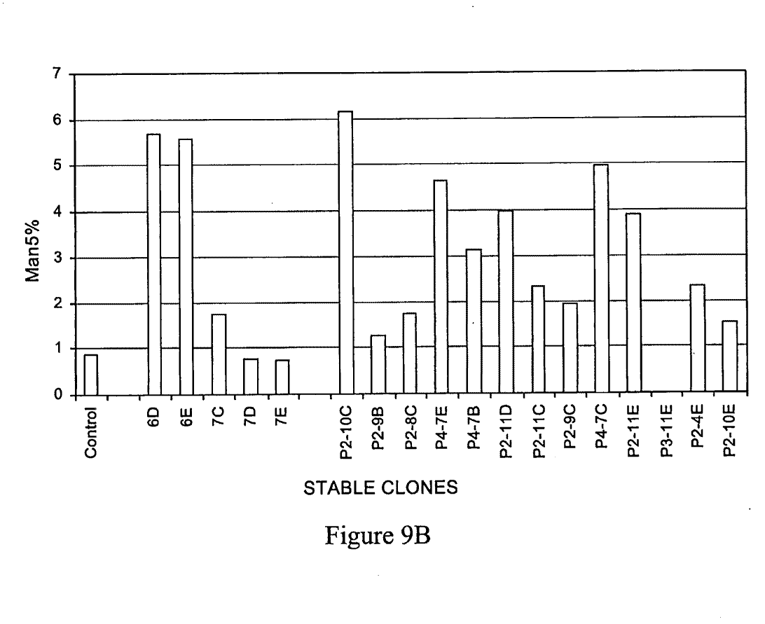

Multiple transfection experiments were performed to create a larger number of stable clones for screening. A total of ˜350 clones were screened using the TAQMAN® assay to determine endogenous mRNA level of GnT-I, where the percentage of mRNA level is relative to the GnT-I mRNA level in the untransfected cell line. After several rounds of scale-up, the top 5 clones from one transfection experiment and the top 13 clones from another transfection experiment were selected, and their relative GnT-I mRNA levels are shown in FIG. 9A. The GnT-I knockdown levels in the stable clones are very similar to the knockdown level observed with transient transfection, with maximum knockdown at 80%. The 18 clones were further evaluated in a 14-day production run, and then the HCCF was analyzed at the end of the run using CE-glycan analysis. The Man5 levels are shown in FIG. 9B. Again the results indicated that the percentage of Man5 glycoform (Man5%) of the stable clones is similar to those obtained with the transient transfection experiment. A roughly 5-fold increase in Man5 level was observed, with the highest level of Man5 at 6% for clone P2-10C.

### EXAMPLE 5

### Manipulating Cell Culture Conditions to Increase Man5 Level

The use of optimized cell culture parameters in conjunction with RNAi knockdown of GnT-I can increase the amount of Man5 obtained. Longer culture duration and increased osmolality media have been found to be beneficial with another antibody evaluated, and results by others (US patent application US2007/0190057-A1 FIG. 2, FIG. 4) have also shown that increasing osmolality can increase the proportion of antibodies with high mannose glycoforms.

FIG. 10A is an example of a production run of the antibody evaluated, which clearly shows that a large amount of Man5 antibodies were produced toward the end of the 14 days culture. In addition, increased NaCl (or osmolality) concentration in basal media was also tested with respect to level of Man5. As shown in FIG. 10B, increasing basal osmolality from 300 to 400 mOsm can further increase Man5 content. However, the addition of high osmolality nutrient supplement solution does not enhance the Man5 level beyond the benefit of the high osmolality basal media (data not shown). The high osmolality and longer culture duration effect can be used in combination in order to increase the Man5 level for other molecules. Due to these findings, an experiment was designed to test these conditions with the cell line generating ocrelizumab and the top 5 GnT-I knockdown stable clones of ocrelizumab described in the previous section.

In addition to the effect caused by osmolality and culture duration, the addition of manganese has been shown to reduce the Man5 level when a small amount of manganese chloride was fed into the culture. FIG. 10C summarizes the results of a 14 day production run with the same antibody, where 1 μM of manganese chloride was fed on either day 3, day 3 & 6, or day 3, 6, &9. In all cases, the Man5 level was decreased by 50% compared to the control. To increase the Man5 level, conditions which lower manganese concentration would be expected to be beneficial.

The top 5 stable clones generated by RNAi knockdown of GnT-I activity were included in this experiment. An example of the results from clone 6D are shown in FIG. 10D. In general, Man5 level increases as culture duration increases for all conditions. High osmolality in basal media appears to have the strongest effect in enhancing the Man5 level, and the absence of manganese has a slight benefit as compared to the control. By extending the production culture from 14 days to 21 days and the usage of high osmolality basal media, the Man5 level can be increased up to 2-fold. Therefore, by manipulating cell culture conditions, the Man5 level can be further enhanced in conjunction with the RNAi knockdown approach.

### EXAMPLE 6

### Use of Lectins to Bind to and Kill Cells Bearing Glycans Produced Downstream of GnT-I

Other methods that result in diminished GnT-I activity in the cell may be used separately or in combination with GnT-I knockdown. Cell lines with a high level of Man5 can also be selected by screening for cell clones with GnT-I mutation, which would lead to activity loss of GnT-I and accumulation of Man5 glycoform. Lectin-resistant methods have been studied by Stanley et al. (Stanley et al., Proc. Nat. Acad. Sci. USA, 72 (9): 3323-3327 (1975); Patnaik and Stanley, Methods Enzymol., 416:159-182 (2006)). For example, a lectin which binds to glycans which are generated downstream of GnT-I can select for cells having a high level of RNAi knockdown. Phytohemagglutinin (PHA), a toxic plant lectin, can be added in cell culture in order to select for cells with low amounts of complex glycans. Cells which lack GnT-I activity will result in defective lectin-binding glycoproteins present on the cell surface, which in turns allow the cells to survive in a PHA-containing environment. This approach can be used in conjunction with RNAi knockdown of GnT-I in order to increase the probability of cells survived under the lectin stress condition. This can also increase the efficiency of finding mutants with a high level of knockdown.

### EXAMPLE 7

### Knock Down of UDP-GlcNAc Golgi Membrane Transporter

Alternatively, knocking down or knocking out one or more additional genes are expected to increase the percentage of Man5. GnT-I requires UDP-GlcNAc as a substrate. UDP-GlcNAc is synthesized in the cytosol, and transported to the lumen of the golgi. Guillen et al (PNAS 95: 7888-7892, 1998) cloned the mammalian Golgi membrane transporter. Knocking down or knocking out this transporter is expected to eliminate or greatly diminish the pool of UDP-GlcNAc in the Golgi apparatus. Accordingly, reducing the level of a substrate for GnT-I, UDP-GlcNAc, is expected to result in higher Man5 levels.

### EXAMPLE 8

Purification and Characterization of Antibodies Bearing Varying Amounts of Man5 Glycans

Antibody enriched in the Man5 glycoform was purified by Con A Sepharose chromatography from harvested, clarified cell culture fluid (HCCF) from a CHO cell fermentation of a humanized IgG1 which binds to a soluble receptor. The cell line expressing this antibody produced a higher than usual amount of Man5 bearing glycans (5-20%).

2 L of HCCF (1.29 g/L mAb) was purified on a PROSEP™ A column (2.5×14 cm, Millipore) equilibrated in 25 mM Tris, 25 mM NaCl, 5 mM EDTA pH 7.1. After a series of post load wash steps using equilibration buffer and a 0.4M Potassium Phosphate buffer, bound antibody was eluted using 0.1M Acetic Acid, pH 2.9, and adjusted to pH 7.4 with 1.5M Tris base. The eluted protein A pool was then processed over a Con A SEPHAROSE™ column (2.5×5 cm, GE Healthcare), equilibrated in 1 mM MnCl₂, 1 mM CaCl₂, 0.5 M NaCl, 25 mM Tris, pH 7.4. Bound antibody was eluted with 0.5M alpha-D-mannopyranoside, 0.5 M NaCl, 25 mM Tris, pH 7.4.

Antibody in the Con A SEPHAROSE™ pool was recovered on the protein A column, and then subjected to chromatography on Con A SEPHAROSE™ a second time. After recovery on protein A, the pool was rechromatographed on Con A SEPHAROSE™ a third time, and this time elution was carried out with a 15 column volume gradient of equilibration buffer and elution buffer. The product was again isolated by protein A chromatography.

Glycan analysis revealed that the starting material contained 15% Man5 glycoform. After 1 pass on Con A, Man5 content increased to 43%, after the second pass Man5 increased to 57%, and to 62% Man5 after the third pass.

Two samples of unenriched (5% Man5 and 16% Man5) antibody and one sample of Con A enriched antibody (62%) were evaluated for Fc gamma receptor IIIa binding by ELISA, and compared to RITUXAN® (rituximab) and HERCEPTIN® (trastuzumab).

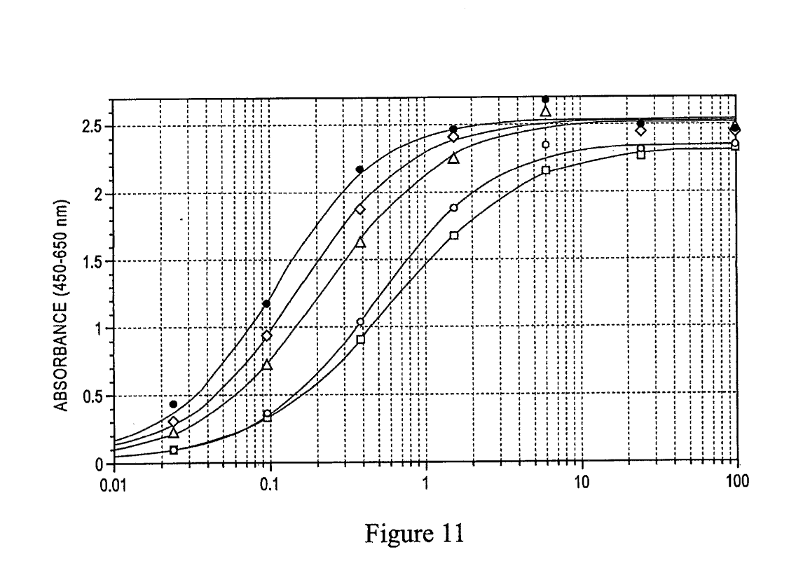

FIG. 11 shows antibody binding to Fc gamma receptor IIIa-V158. Open circles represent HERCEPTIN® (trastuzumab, open squares represent RITUXAN® (rituximab), open triangles represent anti-receptor antibody with 5% Man5 (7-9% afucosyl glycans), open diamonds represent anti-receptor antibody with 16% Man5 (14.6% afucosyl glycans), and closed circles represent anti-receptor antibody with 62% Man5 (11% afucosyl glycans).

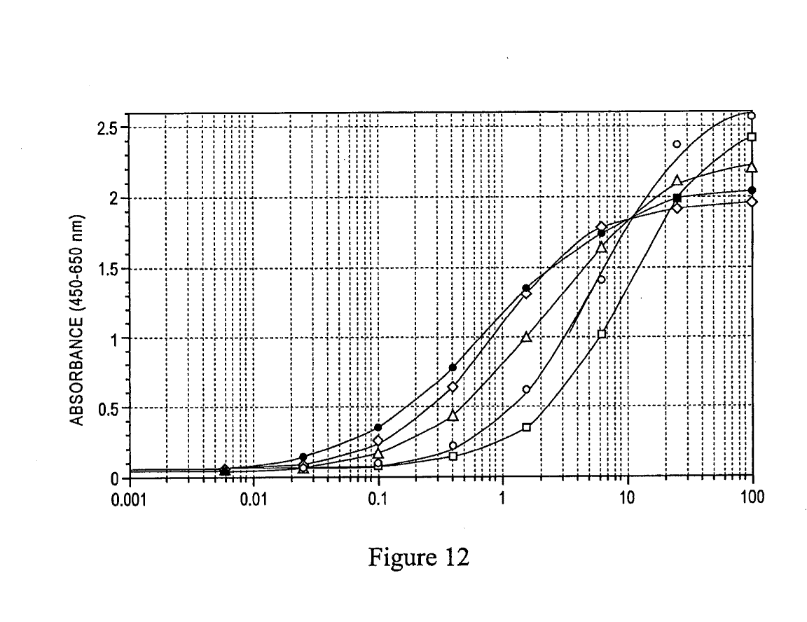

FIG. 12 shows antibody binding to Fc gamma receptor IIIa-F158. Open circles represent HERCEPTIN® (trastuzumab), open squares represent RITUXAN® (rituximab), open triangles represent anti-receptor antibody with 5% Man5 (7-9% afucosyl glycans), open diamonds represent anti-receptor antibody with 16% Man5 (14.6% afucosyl glycans), and closed circles represent anti-receptor antibody with 62% Man5 (11% afucosyl glycans).

The Fc gamma receptor binding assay data (relative affinity) are summarized in the following Table.

| Sample | RIIIa (F158) | RIIIa (V158) |
|---|---|---|
| RITUXAN ® | 1.0 | 1.0 |
| HERCEPTIN ® | 1.81 | 1.32 |
| mAb with 5% | 5.10 | 2.78 |
| Man5 | | |
| mAb with 16% | 11.54 | 4.26 |
| Man5 | | |
| mAb with 62% | 12.72 | 7.03 |
| Man5 | | |

Throughout the foregoing description the invention has been discussed with reference to certain embodiments, but it is not so limited. Indeed, various modifications of the invention in addition to those shown and described herein will become apparent to those skilled in the art from the foregoing description and fall within the scope of the appended claims.

What is claimed is:

1. A mammalian cell lacking GlcNAc Transferase I activity, engineered to express an antibody or a fragment thereof, or an immunoadhesin or a fragment thereof wherein said fragment comprises at least one glycosylation site.

2. The mammalian cell of claim 1 additionally having enhanced α-1,2-mannosidase activity.

3. The mammalian cell of claim 2 which is a cell line.

4. The mammalian cell of claim 3, which is a Chinese Hamster Ovary (CHO) cell line.

5. The mammalian cell of claim 3, wherein the antibody or antibody fragment binds to an antigen selected from the group consisting of CD3, CD4, CD8, CD19, CD20, CD22, CD34, CD40, EGF receptor (EGFR, HER1, ErbB1), HER2 (ErbB2), HER3 (ErbB3), HER4 (ErbB4), LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DR5, EGFL7, neuropolins and receptors thereof, VEGF-C, ephrins and receptors thereof, netrins and receptors thereof, slit and receptors thereof, sema and receptors thereof, semaphorins and receptors thereof, robo and receptors thereof, and M1.

6. The mammalian cell of claim 5 wherein said antibody is chimeric or humanized.

7. The mammalian cell of claim 6 wherein the chimeric antibody is an anti-CD20 antibody.

8. The mammalian cell of claim 7 wherein the anti-CD20 antibody is rituximab or ocrelizumab.

9. The mammalian cell of claim 6 wherein the humanized antibody is an anti-HER2, anti-HER1, anti-VEGF or anti-IgE antibody.

10. The mammalian cell of claim 9 wherein the anti-HER2 antibody is trastuzumab or pertuzumab.

11. The mammalian cell of claim 9 wherein the anti-VEGF antibody is bevacizumab, or ranibizumab.

12. The mammalian cell of claim 9 wherein the anti-IgE antibody is omalizumab.

13. The mammalian cell of claim 5 wherein the antibody fragment is selected from the group consisting of complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide.

14. A mammalian cell, in which GlcNAc Transferase I activity is diminished by RNAi knockdown, engineered to express an antibody or a fragment thereof, or an immunoadhesin or a fragment thereof, wherein said fragment comprises at least one glycosylation site.

15. The mammalian cell of claim 14, in which GlcNAc Transferase I activity is diminished by RNAi knockdown, sufficient to result in a carbohydrate structure comprising 20% or greater Man5, Man6 glycans.

16. The mammalian cell of claim 14, in which GlcNAc Transferase I activity is diminished by RNAi knockdown, sufficient to result in a carbohydrate structure comprising 25% or greater Man5, Man6 glycans.

17. The mammalian cell of claim 14, additionally having enhanced α-1,2-mannosidase activity.

18. The mammalian cell of claim 17, engineered to express an antibody or a fragment thereof, or an immunoadhesin or a fragment thereof, wherein said antibody or fragment thereof comprises a carbohydrate structure of 20% or greater Man5, Man6 glycans.

19. The mammalian cell line of claim 17, engineered to express an antibody or a fragment thereof, or an immunoadhesin or a fragment thereof, wherein said antibody or fragment thereof comprises a carbohydrate structure of 25% or greater Man5, Man6 glycans.

20. The mammalian cell of claim 17 which is a cell line.

21. The mammalian cell of claim 20, which is a Chinese Hamster Ovary (CHO) cell line.

22. The mammalian cell of claim 17, wherein the antibody or antibody fragment binds to an antigen selected from the group consisting of CD3, CD4, CD8, CD19, CD20, CD22, CD34, CD40, EGF receptor (EGFR, HER1, ErbB1), HER2 (ErbB2), HER3 (ErbB3), HER4 (ErbB4), LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DRS, EGFL7, neuropolins and receptors thereof, VEGF-C, ephrins and receptors thereof, netrins and receptors thereof, slit and receptors thereof, sema and receptors thereof, semaphorins and receptors thereof, robo and receptors thereof, and M1.

23. The mammalian cell of claim 14 wherein said antibody is chimeric or humanized.

24. The mammalian cell of claim 23 wherein the chimeric antibody is an anti-CD20 antibody.

25. The mammalian cell of claim 24 wherein the anti-CD20 antibody is rituximab or ocrelizumab.

26. The mammalian cell of claim 23 wherein the humanized antibody is an anti-HER2, anti-HER1, anti-VEGF or anti-IgE antibody.

27. The mammalian cell of claim 26 wherein the anti-HER2 antibody is trastuzumab or pertuzumab.

28. The mammalian cell of claim 26 wherein the anti-VEGF antibody is bevacizumab, or ranibizumab.

29. The mammalian cell of claim 26 wherein the anti-IgE antibody is omalizumab.

30. The mammalian cell of claim 26 wherein the antibody fragment is selected from the group consisting of complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide.

31. A mammalian cell, in which GlcNAc Transferase I activity is diminished by RNAi knockdown of the Golgi UDP-GlcNAc transporter, engineered to express an antibody or a fragment thereof, or an immunoadhesin or a fragment thereof, wherein said fragment comprises at least one glycosylation site.

32. The mammalian cell of claim 31, wherein the mammalian cell additionally has enhanced α-1,2-mannosidase activity.

33. A mammalian cell, in which GlcNAc Transferase I activity is diminished by RNAi knockdown of the Golgi UDP-GlcNAc transporter, and which also has GlcNAc transferase I knocked down by RNAi, engineered to express an antibody or a fragment thereof, or an immunoadhesin or a fragment thereof, wherein the fragment comprises at least one glycosylation site.

34. The mammalian cell of claim 33, wherein the mammalian cell additionally has enhanced α-1,2-mannosidase activity.

35. A method for making an antibody or a fragment thereof, or an immunoadhesin or a fragment thereof, bearing predominantly Man5 glycans, comprising culturing a mammalian cell line according to claim 3 or claim 20 under conditions such that said antibody or a fragment thereof, or an immunoadhesin or a fragment thereof is produced, wherein said fragment comprises at least one glycosylation site.

36. The method of claim 35 wherein the mammalian cell line is a Chinese Hamster Ovary (CHO) cell line, wherein the antibody or fragment thereof, or the immunoadhesin or fragment thereof, bear 20% or greater Man5 glycans.

37. The method of claim 35 wherein the mammalian cell line is a Chinese Hamster Ovary (CHO) cell line, wherein the antibody or fragment thereof, or the immunoadhesin or fragment thereof, bear 25% or greater Man5 glycans.

38. The method of claim 35 wherein the mammalian cell line is a Chinese Hamster Ovary (CHO) cell line, wherein the antibody or fragment thereof, or the immunoadhesin or fragment thereof, bear 30% or greater Man5 glycans.

39. The method of claim 35 wherein the mammalian cell line is a Chinese Hamster Ovary (CHO) cell line, wherein the antibody or fragment thereof, or the immunoadhesin or fragment thereof, bear 35% or greater Man5 glycans.

40. The method of claim 35, wherein the antibody or antibody fragment binds to an antigen selected from the group consisting of CD3, CD4, CD8, CD19, CD20, CD22, CD34, CD40, EGF receptor (EGFR, HER1, ErbB1), HER2 (ErbB2), HER3 (ErbB3), HER4 (ErbB4), LFA-1, Mac1, p150,95, VLA-4, ICAM-1, VCAM, αv/β3 integrin, CD11a, CD18, CD11b, VEGF; IgE; blood group antigens; flk2/flt3 receptor; obesity (OB) receptor; mpl receptor; CTLA-4; protein C, DRS, EGFL7, neuropolins and receptors thereof, VEGF-C, ephrins and receptors thereof, netrins and receptors thereof, slit and receptors thereof, sema and receptors thereof, semaphorins and receptors thereof, robo and receptors thereof, and anti-M1.

41. The method of claim 40 wherein said antibody is chimeric or humanized.

42. The method of claim 41 wherein the chimeric antibody is an anti-CD20 antibody.

43. The method of claim 42 wherein the anti-CD20 antibody is rituximab or ocrelizumab.

44. The method of claim 41 wherein the humanized antibody is an anti-HER2, anti-HER1, anti-VEGF or anti-IgE antibody.

45. The method of claim 44 wherein the anti-HER2 antibody is trastuzumab or pertuzumab.

46. The method of claim 44 wherein the anti-VEGF antibody is bevacizumab, or ranibizumab.

47. The method of claim 44 wherein the anti-IgE antibody is omalizumab.

48. The method of claim 40 wherein the antibody fragment is selected from the group consisting of complementarity determining region (CDR) fragments, linear antibodies, single-chain antibody molecules, minibodies, diabodies, multispecific antibodies formed from antibody fragments, and polypeptides that contain at least a portion of an immunoglobulin that is sufficient to confer specific antigen binding to the polypeptide.

50. The method of claim 35, comprising culturing said mammalian cell line lacking GlcNAc Transferase I activity engineered to express said antibody, immunoadhesin, or fragment thereof in the presence of an α-1,2-mannosidase, or contacting the expressed product with such α-1,2-mannosidase, wherein Man7,8,9 glycans are converted to Man5 glycans, wherein said fragment comprises at least one glycosylation site.

51. A method for recombinant production of an antibody, an immunoadhesin, or a fragment thereof with about 20% to 100% Man5 glycans in the carbohydrate structure thereof, comprising expressing nucleic acid encoding said antibody or antibody fragment in a mammalian cell line which has a diminished GlcNAc Transferase I activity as a result of RNAi knockdown, wherein said fragment comprises at least one glycosylation site.

52. A method for recombinant production of an antibody, an immunoadhesin, or a fragment thereof, bearing predominantly Man5 glycans in the carbohydrate structure thereof, comprising culturing a mammalian cell line with diminished GcNAn Transferase I activity due to RNAi knockdown, engineered to express said antibody, immunoadhesin, or a fragment thereof, wherein Man7,8,9 glycans are converted to Man5 glycans, wherein said fragment comprises at least one glycosylation site.

53. The method of claim 52 further comprising culturing a mammalian cell line with diminished GcNAn Transferase I activity due to RNAi knockdown, engineered to express said antibody, immunoadhesin, or a fragment thereof, in the presence of an α-1,2-mannosidase, or contacting the expressed product with such α-1,2-mannosidase, wherein Man7,8,9 glycans are converted to Man5 glycans, wherein said fragment comprises at least one glycosylation site.

54. A method for recombinant production of an antibody, an immunoadhesin, or a fragment thereof, bearing predominantly Man5 glycans in the carbohydrate structure thereof, comprising culturing mammalian cells in the presence of a toxic lectin to select for clones with diminished GlcNAc Transferase I activity, and engineering one or more of said clones with diminished GlcNAc Transferase I activity to express said antibody, immunoadhesin, or a fragment thereof, wherein Man7,8,9 glycans are converted to Man5 glycans, and wherein said fragment comprises at least one glycosylation site.

55. The method of claim 54 wherein the toxic lectin is phytohemagglutinin.

56. The method of claim 54 wherein the selection of clones with diminished GlcNAc Transferase I activity is used to identify cells in which GlcNAc Transferase I activity has been diminished by RNAi knockdown.

57. The method of claim 54 further comprising culturing mammalian cells in the presence of an α-1,2-mannosidase, or contacting the expressed product with such α-1,2-mannosidase, wherein Man7,8,9 glycans are converted to Man5 glycans, and wherein said fragment comprises at least one glycosylation site.

58. A method for recombinant production of an antibody, an immunoadhesin, or a fragment thereof, bearing predominantly Man5 glycans in the carbohydrate structure thereof, comprising culturing a mammalian cell line lacking UDP-GlcNAc transporter activity engineered to express said antibody, immunoadhesin, or fragment thereof, or contacting the expressed product with such α-1,2-mannosidase, wherein Man7,8,9 glycans are converted to Man5 glycans, wherein said fragment comprises at least one glycosylation site.

59. The method of claim 58 further comprising culturing mammalian cells in the presence of an α-1,2-mannosidase, or contacting the expressed product with such α-1,2-mannosidase, wherein Man7,8,9 glycans are converted to Man5 glycans, and wherein said fragment comprises at least one glycosylation site.

60. The method of claim 58 wherein an endogenous mannosidase activity in the cell is used for recombinant production of antibodies or fragments thereof.


SEQUENCE LISTING (13 sequences)

SEQ ID NO: 1 - protein, 9 aa, Artificial Sequence
  source: /note=“Description of Artificial Sequence Synthetic peptide”
MDYKDDDDK

SEQ ID NO: 2 - DNA, 126 bp, Cricetulus griseus
gatccgttccagggttactacaagttcaagagacttgtagtaaccctggaacttttttggaaagcaaggtcccaatgatgttcaagttctctgaacatcattgggaccttgaaaaaaccttttcga

SEQ ID NO: 3 - DNA, 126 bp, Cricetulus griseus
gatccatcagggagcatcatgcttctcaagagaaagcatgatgctccctgatttttttggaaagtagtccctcgtagtacgaagagttctctttcgtactacgagggactaaaaaaaccttttcga

SEQ ID NO: 4 - DNA, 128 bp, Cricetulus griseus
gatccgtgacaatggcaaggagcagttcaagagactgctccttgccattgtcattttttggaaagcactgttaccgttcctcgtcaagttctctgacgaggaacggtaacagtaaaaaaccttttcga

SEQ ID NO: 5 - DNA, 126 bp, Cricetulus griseus
gatccgttcatcaagctgaaccagttcaagagactggttcagcttgatgaacttttttggaaagcaagtagttcgacttggtcaagttctctgaccaagtcgaactacttgaaaaaaccttttcga

SEQ ID NO: 6 - DNA, 126 bp, Cricetulus griseus
gatccggctatgatcctagctggattcaagagatccagctaggatcatagccttttttggaaagccgatactaggatcgacctaagttctctaggtcgatcctagtatcggaaaaaaccttttcga

SEQ ID NO: 7 - DNA, 21 bp, Artificial Sequence
  source: /note=“Description of Artificial Sequence Synthetic primer”
cgttgtcactttccagttcag

SEQ ID NO: 8 - DNA, 18 bp, Artificial Sequence
  source: /note=“Description of Artificial Sequence Synthetic primer”
agccttcccaggtttgtg

SEQ ID NO: 9 - DNA, 20 bp, Artificial Sequence
  source: /note=“Description of Artificial Sequence Synthetic probe”
acgtgtccacctggcacccc

SEQ ID NO: 10 - RNA, 49 nt, Artificial Sequence
  source: /note=“Description of Artificial Sequence Synthetic oligonucleotide”
nnnnnnnnnnnnnnnnnnnuucaagagannnnnnnnnnnnnnnnnnnuu

SEQ ID NO: 11 - DNA, 1968 bp, Cricetulus griseus
atgcccgtggggggcctgttgccgctcttcagtagcccggggggcggcggcctgggcggcggcctgggcggggggcttagtggcagtagaaaagggtctggccccgcagccttccgcctcaccgagaagttcgtgttgctgctggtgttcagcgccttcatcacgctctgcttcggggcaatcttcttcctgcctgactcctccaagctgctcagcggggtcctgttccactccaaccccgccttgcagccggcggcagagcacaagcccgggcccggggcgcgtgccgaagatgccgccgaggggcgagtccggcaccgcgaggaaggcgtgcccggggaccccggggctgtagtggaggacaacttagccaggatccgtgaaaaccacgagcgggctctcagggaagccaaggagaccctgcagaagctgcccgaagaaattcaaagagacattctgctggagaaggaaaaggtggcccaggaccatatgcgtgacaaggagctgtttgggggcctgcccaaggtggacttcctacctcccatcggggtagagaaccgagagccagctgatgccaccatccgcgagaagagagcaaagatcaaagagatgatgaaccatgcttggaataattataaacgctatgcctggggcttaaatgaactgaagcctatatcaaaagaaggccattcaagcagtttatttggcaacatcaaaggagcaacaatcgtagatgccctggatacacttttcattatgggaatgaagactgaatttcaggaagctaaatcatggattaaaaaatatttagattttaatgtgaatgctgaagtttctgtttttgaagtaaatatacgcttcgtcggtggactgctgtcagcctactacttgtctggggaagagatatttcgaaagaaagcagtggaacttggggtaaaattgctacctgcatttcatactccctctggaataccttgggcattgctgaatatgaaaagtggtattggacggaactggccctgggcctctggaggcagcagtattctggcagaatttggaactttgcatttggagttcatgcacttgagccacttatctggaaaccccatcttcgctgaaaaagtaatgaatattcgaacagtgctgaacaaactggaaaaaccagaaggcctttatcctaactatctgaaccccagcagtgggcagtggggtcaacatcatgtgtcagttggaggacttggagacagcttttatgaatatctgctcaaggcatggttaatgtctgacaagaccgatgtagaagccaagaagatgtattttgatgctgttcaggccattgagactcacctgatccgcaagtccagcgggggactaacatacatcgcagagtggaaaggaggcctcctggaacacaaaatgggccacctgacctgctttgcagggggtatgtttgcccttggggcagatggagctcccgaagccctggctcaacactaccttgaactcggtgctgaaatcgcacgcacctgtcatgaatcttacaatcgcaccttcatgaagctgggaccagaagctttccgatttgatggcggtgtggaagccattgccacgaggcaaaatgaaaagtactacatcttacggcctgaagtcatcgagacttacatgtacatgtggcgactgactcatgaccccaagtacagagcctgggcctgggaagccgtagaggccctagaaaaccactgccgagtgaacggaggctactcgggcctacgggatgtttactttgctagtgagagttatgacgatgtccagcaaagtttcttcctggcagagacactaaagtatttgtacttgatattttctgatgatgaccttcttccactagaacactgggtcttcaatactgaggcacaccctttccccatactccgagacgagaaaaaggaaattgaagtcaaagagaaatga

SEQ ID NO: 12 - protein, 640 aa, Mus sp.
GGLGSGLGGGLGGGRKGSGPAAFRLTEKFVLLLVFSAFITLCFGAIFFLPDSSKLLSGVLFHSNPALQPPAEHKPGLGARAEDAAEGRVRHREEGAPGDPGAGLEDNLARIRENHERALREAKETLQKLPEEIQRDILLEKEKVAQDQLRDKDLFRGLPKVDFLPPVGVENREPADATIREKRAKIKEMMTHAWNNYKRYAWGLNELKPISKEGHSSSLFGNIKGATIVDALDTLFIMGMKTEFQEAKSWIKKYLDFNVNAEVSVFEVNIRFVGGLLSAYYLSGEEIFRKKAVELGVKLLPAFHTPSGIPWALLNMKSGIGRNWPWASGGSSILAEFGTLHLEFMHLSHLSGDPVFAEKVMKIRTVLNKLDKPEGLYPNYLNPSSGQWGQHHVSVGGLGDSFYEYLLKAWLMSDKTDLEAKKMYFDAVQAIETHLIRKSSGGLTYIAEWKGGLLEHKMGHLTCFAGGMFALGADGAPEARAQHYLELGAEIARTCHESYNRTYVKLGPEAFRFDGGVEAIATRQNEKYYILRPEVIETYMYMWRLTHDPKYRTWAWEAVEALESHCRVNGGYSGLRDVYIARESYDDVQQSFFLAETLKYLYLIFSDDDLLPLEHWIFNTEAHPFPILREQKKEIDGKEK

SEQ ID NO: 13 - protein, 640 aa, Cricetulus griseus
GGLGGGLGGGLSGSRKGSGPAAFRLTEKFVLLLVFSAFITLCFGAIFFLPDSSKLLSGVLFHSNPALQPAAEHKPGPGARAEDAAEGRVRHREEGVPGDPGAVVEDNLARIRENHERALREAKETLQKLPEEIQRDILLEKEKVAQDHMRDKELFGGLPKVDFLPPIGVENREPADATIREKRAKIKEMMNHAWNNYKRYAWGLNELKPISKEGHSSSLFGNIKGATIVDALDTLFIMGMKTEFQEAKSWIKKYLDFNVNAEVSVFEVNIRFVGGLLSAYYLSGEEIFRKKAVELGVKLLPAFHTPSGIPWALLNMKSGIGRNWPWASGGSSILAEFGTLHLEFMHLSHLSGNPIFAEKVMNIRTVLNKLEKPEGLYPNYLNPSSGQWGQHHVSVGGLGDSFYEYLLKAWLMSDKTDVEAKKMYFDAVQAIETHLIRKSSGGLTYIAEWKGGLLEHKMGHLTCFAGGMFALGADGAPEALAQHYLELGAEIARTCHESYNRTFMKLGPEAFRFDGGVEAIATRQNEKYYILRPEVIETYMYMWRLTHDPKYRAWAWEAVEALENHCRVNGGYSGLRDVYFASESYDDVQQSFFLAETLKYLYLIFSDDDLLPLEHWVFNTEAHPFPILRDEKKEIEVKEK